(19) United States
(12) Patent Application Publication
(10) Pub. No.: US 2019/0298176 A1
(43) Pub. Date: Oct. 3, 2019

(54) METHOD OF CAPTURING DIGITAL OCCLUSAL BITE DATA FOR EDENTULOUS PATIENTS

(71) Applicants: Luiz Alfredo Dos-Santos, Las Vegas, NV (US); Shawn S. Ahn, Las Vegas, NV (US)

(72) Inventors: Luiz Alfredo Dos-Santos, Las Vegas, NV (US); Shawn S. Ahn, Las Vegas, NV (US)

(21) Appl. No.: 16/281,511

(22) Filed: Feb. 21, 2019

Related U.S. Application Data
(60) Provisional application No. 62/649,662, filed on Mar. 29, 2018.

(51) Int. Cl. A61B 5/00; A61C 9/00

(52) U.S. Cl. CPC A61B 5/0088; A61C 9/0053; A61B 5/004

(57) ABSTRACT

A method of obtaining a digital occlusal bite, whether of single or both edentulous arches, without the need for a dental/arch impression. The method may include creating a clear stent from a patient's existing denture, placing one or more abutments into position according to the clear stent, scanning one or more of the patient's dental arches while one or more abutments are in place, and placing the stent in the patient's mouth into a bite position for capturing a true occlusal bite as a digital file.

### CROSS-REFERENCE TO RELATED APPLICATION

This application claims the benefit of co-pending U.S. Provisional Patent Application Ser. No. 62/649,662, filed Mar. 29, 2018, which is incorporated herein in its entirety.

### FIELD OF THE INVENTION

The present invention relates generally to dental analysis and more particularly to a method of capturing digital occlusal bite data for edentulous patients and exposing and analyzing tissue complications within the mouth.

### BACKGROUND OF THE INVENTION

It is common for a dentist to analyze a patient's bite for dental prostheses fabrication. For example, to prepare fixed implant prostheses for a patient, dimensions of the patient's dental arches must be carefully and accurately taken into account.

There are different prostheses for replacing teeth of an edentulous patient. For example, a removal denture is a most commonly used prosthetic device to replace patient's missing teeth. Other prostheses include removable implant supported dentures and fixed implant supported dental prostheses.

There exists well documented literature on dentures and implant supported dentures for taking physical impressions to create a laboratory models. With this method, in order to establish jaw relation records (e.g. occlusal bite), patients are required to come in to a dental office for an inconvenient series of appointments for impressions and try-ins before fabrication of a final prosthesis by a dental laboratory. Shipping the restorative components from the dental office to the dental laboratory makes this process additionally time consuming and very expensive. Another problem encountered with physical impressions is the possibility of cumulative error in accuracy during impression taking, and laboratory model fabrication.

Therefore, there exists a need for an efficient and effective method for recording an occlusal bite of an edentulous patient for fabricating a fixed dental prosthesis without the need for taking physical impressions.

### SUMMARY OF THE INVENTION

This summary is provided to introduce a selection of concepts in a simplified form that are further described below in the detailed description. This summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

Disclosed is a method for capturing digital occlusal bite data for edentulous patients, the method comprising, creating a clear stent from a patient's existing denture, placing one or more abutments into position according to the clear stent, scanning one or more of the patient's dental arches while one or more abutments are in place, and placing the stent in the patient's mouth into a bite position for capturing a true occlusal bite as a digital file.

Introducing a first embodiment of the invention, the present invention consists of a method comprising:

creating a clear stent from a patient's existing denture;

placing one or more abutments into position according to the clear stent;

scanning one or more of the patient's dental arches while one or more abutments are in place; and

placing the stent in the patient's mouth into a bite position for capturing a true occlusal bite as a digital file.

Introducing a second embodiment of the invention, the present invention consists of a method comprising:
performing a first scanning of the mouth of a patient;
generating and inserting, based on the first scanning, a teeth receptacle;
implanting one or more abutments in the mouth of the patient;
performing a second scan, based on the placement of the teeth receptacle, in the mouth of the patient;
capturing, based on the second scanning, a true occlusal bite as a digital file; and
generating, based on the digital file, a fabricated prosthesis configured to fit the teeth of the patient.

Introducing a third embodiment of the invention, the present invention consists of a method for exposing tissue issues at one or more dental arches comprising:
placing a teeth receptacle in the mouth of a patient;
placing one or more abutments in position based on the teeth receptacle;
scanning the mouth of the patient based on the one or more abutments; and
based on the scanning, capturing a true occlusal bite as a digital file.

In another aspect, the digital file is sent to a laboratory for final fabrication of a fixed dental prosthesis.

In another aspect, the clear stent is configured to expose gingival tissue at the one or more arches for the gingival tissue to be scanned.

In another aspect, the gingival tissue is maxilliary and mandibular tissue.

In another aspect, the clear stent is configured to expose gingival tissue to provide an open scanning path between arches when the clear stent is held in the patient's mouth.

In another aspect, the clear stent includes apertures to expose the gingival tissue to provide a clear scanning path between the arches while the clear stent is held in the patient's mouth in the bite position.

In another aspect, scanning the gingival tissue includes capturing positions of at least one of the abutments to obtain the true bite.

In another aspect, no more than one three dimensional scan image is taken of the maxilliary arch and mandibular arch.

In another aspect; scanning the arches before the clear stent is placed in the patient's mouth in the bite position is a first set of data, and scanning the arches in the bite position is a second set of data, and the true bite is obtained by modeling relative positions of the arches between the first set of data and the second set of data.

These and other objects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 presents a flow chart of a method of capturing digital occlusal bite data for an edentulous patient and sending the bite data to a lab for fabrication of a prosthesis; and

FIG. 2 presents a front view of an edentulous patient's true bite, where a clear stent shaped from the patient's own dentures is being bit down on.

Like reference numerals refer to like parts throughout the several views of the drawings.

### DETAILED DESCRIPTION

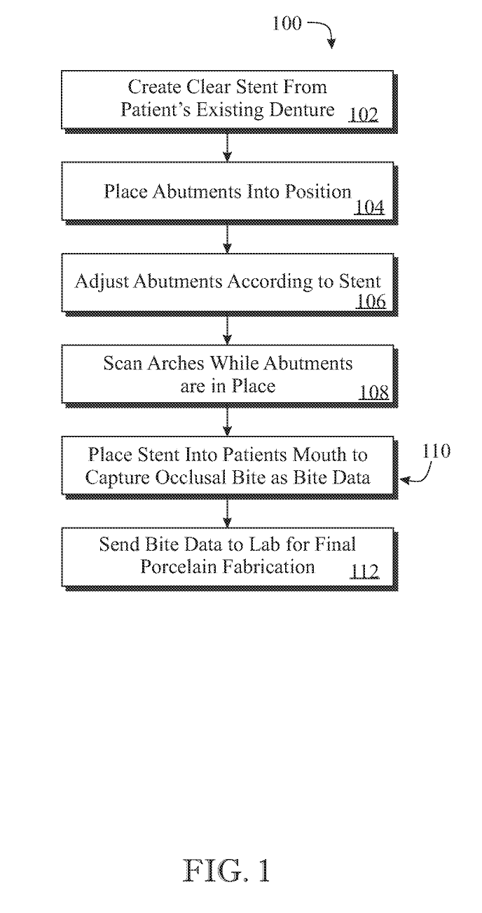

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, “vertical”, “horizontal”, and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Shown throughout the figures, a method for capturing digital occlusal bite data for edentulous patients (method 100, FIG. 1) is disclosed.

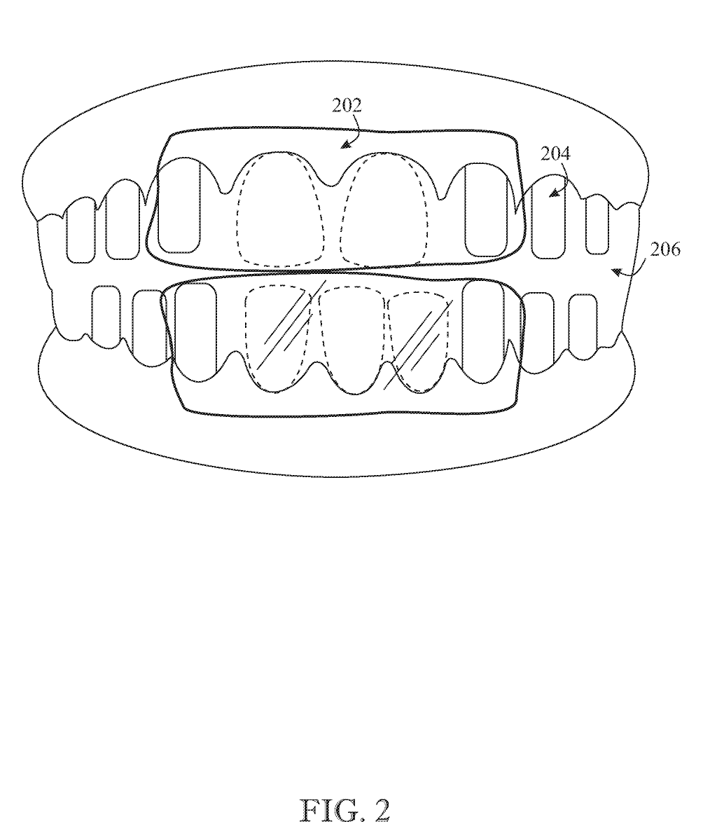

The method 100 may include, at 102, creating a clear stent 202. (FIG. 2) from a patient's existing denture. For example, the clear stent 202 may be molded from a patient's existing denture. After being created, the clear stent 202 may be stiff or rigid enough such that the stent does not bend when a patient gently bites on the clear stent 202. It is to be understood that the clear stent 202 may have any appropriate transparency or rigidity without departing from the spirit and scope of this disclosure. It is to be understood that the clear stent 202 may include one or a plurality of pieces. For example, FIG. 2 shows two pieces of a clear stent 202 occluding on each other. A first piece is located on maxillary teeth and a second piece is located on mandibular teeth for occluding on each other.

Further, it is to be understood that dotted lines shown in FIG. 2 may indicate teeth of a partially edentulous patient, where dotted upper central incisors and lower central and left lateral incisors are shown. Therefore, the teeth shown by the dotted lines may or may not be present, without departing from the spirit or scope of this disclosure.

The method 100 may include, at 104, placing one or more abutments 204 (FIG. 2) into position in a patient's gingival tissue, and at 106 adjusting the abutments 204 according to the clear stent 202. For example, the abutments 204 may be placed to line up with positions of teeth indentations of the clear stent 202. It is to be understood that any number of abutments 204 may be included without departing from the spirit or scope of this disclosure.

Method 100 may include, at 108, scanning one or more of the patient's dental arches while one or more abutments 204 are in place.

It is to be understood that the stent may be clear or transparent enough to facilitate effective scanning. For example, a scan may be an optical intra-oral scan. Translucency of the stent may be selected such that the abutments are optically visible through the stent, and/or such that a color of the stent is not (or minimally) picked up via the scanner.

It is to be understood, that the herein disclosed apertures may generally refer to a space 206 (FIG. 2) that exposes tissue and abutments 204. For example, all three abutments, or all abutments, may be exposed from sides of the patient's arches. The purpose of the stent 202 is to act as a physical structure to guide the user's bite. Since the stent 202 may be picked up inadvertently through the scan, the apertures or spaces 206 allow the abutments to be properly exposed to optical scans. However, in some instances, optical scans of the abutments may be accomplished through the material of the stent 202 if the stent is properly configured and transparent enough.

Method 100 may include, at 110, placing the stent 202 in the patient's mouth into a bite position for capturing a true occlusal bite as a digital file. As a non-limiting example, scanning the arches before the clear stent 202 is placed in the patient's mouth in the bite position may be considered a first set of data, and scanning the arches in the bite position may be considered a second set of data, and the true occlusal bite may obtained by modeling relative positions of the arches between the first set of data and the second set of data. At 112, the digital file may be sent to a laboratory for final fabrication of a fixed dental prosthesis.

As a non-limiting example, the clear stent 202 may be configured to expose gingival tissue at the one or more arches for the gingival tissue to be scanned. As a non-limiting example, the gingival tissue may be maxilliary and mandibular tissue. As a non-limiting example, the clear stent may be configured to expose gingival tissue to provide an open and/or non-interrupted scanning path between arches when or while the clear stent 202 is held in the patient's mouth. As a non-limiting example, the clear stent 202 may include one or more apertures to expose the gingival tissue to provide a clear, open, and/or uninterrupted scanning path between the arches while the clear stent 202 is held in the patient's mouth in the bite position. As shown in FIG. 2, the clear stent 202 may be dimensioned such that space 206 is left between horizontal edges of the clear stent 202 and corners of the patient's mouth. This space allows clear, open, and/or uninterrupted scanning of the arches without any material affecting the scan. As a non-limiting example, scanning the gingival tissue may include capturing positions of one or more of the abutments to obtain the true bite. In one example, no more than one three dimensional scan image is taken of the maxilliary arch and mandibular arch.

As a non-limiting example, the following steps may be followed to implement the disclosed method:

In a first step, dental cone beam computed tomography (CBCT), panoramic x-ray, intra oral scanning, and/or intra-oral imaging may be done. The patient may be consulted.

In a second step, implant planning may be done and a surgical guide may be fabricated.

In a third step, implants may be placed via guided surgery.

In a fourth step, a clear stent may be created from the patient's existing denture/dentures.

In a fifth step, abutments may be placed into position and adjusted according to the clear stent.

In a sixth step, the patient's mouth may be prepared to scan the abutments for final prosthesis.

In a seventh step, arch/arches may be scanned with abutments in place.

In an eighth step, the stent is placed into the patient's mouth to be able to capture the occlusal bite (vertical dimension of occlusion j as a digital file.

In a ninth step, once the bite is captured the digital file a be sent to the laboratory for final porcelain fabrication.

In a tenth step, the patient may be invited back to the office once the lab has fabricated the patients prosthesis according to the digital file.

In an eleventh step, the implant abutments are placed back into the patient's mouth and the prosthesis is loaded over the abutments.

In a twelfth step, minor adjustments are made to the above described components.

In a thirteenth step, final panoramic x-rays, cephalometric x-rays, and intra oral imaging may be taken and the case finalized. In conclusion the disclosed method allows a dental professional to obtain a digital occlusal bite, whether of single or both edentulous arches, without the need for a dental/arch impression.

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A method for capturing digital occlusal bite data, the method comprising:
creating a transparent stent from a patient's existing denture;
placing one or more abutments into position according to the transparent stent;
scanning one or more of the patient's dental arches while one or more abutments are in place; and
placing the stent the patient's mouth into a bite position for capturing a true occlusal bite as a digital file.

2. The method of claim 1, wherein the digital file is sent to a laboratory for final fabrication of a fixed dental prosthesis.

3. The method of claim 1, wherein the transparent stent is configured to expose gingival tissue at the one or more arches for the gingival tissue to be scanned.

4. The method of claim 1, wherein the gingival tissue is at least one of maxilliary and mandibular tissue.

5. The method of claim 1, wherein the transparent stent is configured to expose gingival tissue to provide an open scanning path between arches when the transparent stent is held in the patient's mouth.

6. The method of claim 1, wherein the transparent stent includes apertures to expose the gingival tissue to provide a clear scanning path between the arches while the transparent stent is held in the patient's mouth in the bite position.

7. The method of claim 1, wherein scanning the gingival tissue includes capturing positions of at least one of the abutments to obtain the true bite.

8. The method of claim 1, wherein no more than one three dimensional scan image is taken of the maxilliary arch or mandibular arch.

9. The method of claim 1, wherein scanning the arches before the transparent stent is placed in the patient's mouth in the bite position is a first set of data, and scanning the arches in the bite position is a second set of data, and the true bite is obtained by modeling relative positions of the arches between the first set of data and the second set of data.

10. A method for capturing digital occlusal bite data, the method comprising:
creating a transparent stent from a patient's existing denture;
placing one or more abutments into position according to the transparent stent;
scanning one or more of the patient's dental arches while one or more abutments are in place;
placing the stent in the patient's mouth into a bite position for capturing a true occlusal bite as a digital file; and
generating, based on the digital file, a fixed dental prosthesis configured to be affixed to teeth of the patient.

11. The method of claim 10, wherein the transparent stent comprises one or more apertures configured to generate one or more scannable passageways between the dental arches.

12. The method of claim 10, wherein the digital file comprises a first set of data comprising a first plurality of dimensions of the dental arches according to absence of the stent, and a second set of data comprising a second plurality of dimensions of the dental arches according to presence of the stent.

13. The method of claim 12, wherein capturing the true occlusal bite comprises comparing the first set of data and the second set of data, and generating an aggregated set of data.

14. The method of claim 10, wherein the transparent stent comprises a first teeth receptacle configured to fit a mandibular set of teeth and a second teeth receptacle configured to fit a maxillary set of teeth.

15. A method for capturing digital occlusal bite data, the method comprising:
in a first step executing at least one of dental cone beam computed tomography (CBCT), panoramic x-ray scanning, intra oral scanning, and intra-oral imaging;
in a second step, executing implant planning and fabricating a surgical guide;
in a third step, placing implants via guided surgery;
in a fourth step, creating a clear stent from a patient's existing denture;
in a fifth step, placing abutments into position and adjusting the abutments according to the clear stent's structure;
in a sixth step, preparing the patient's mouth for scanning the abutments for final prosthesis;
in a seventh step, scanning one or both dental arches with abutments in place; and
in an eighth step, placing the stent into the patient's mouth to capture a vertical dimension of occlusion and an occlusal bite as a digital file.

16. The method of claim 15, wherein the method further comprises:
in a ninth step, once the bite is captured, sending the digital file to a laboratory for final porcelain fabrication.

17. The method of claim 15, wherein the method further comprises:
in a tenth step, placing implant abutments back into the patient's mouth and loading the prosthesis over the abutments.

18. The method of claim 15, wherein the method further comprises:
in an eleventh step, making minor adjustments by repeating one or more of the previous steps.

19. The method of claim 15, wherein the method further comprises:
in a twelfth step, at least one of final panoramic x-rays, cephalometric x-rays, and intra oral imaging are taken to finalize the method.

20. The method of claim 15, wherein the method further comprises obtaining a digital occlusal bite of edentulous arches without a dental or arch impression.
